(19) United States
(12) Patent Application Publication
(10) Pub. No.: US 2010/0196273 A1
(43) Pub. Date: Aug. 5, 2010

(54) NOVEL AGENT FOR IN VIVO PET IMAGING OF TUMOR PROLIFERATION

(75) Inventors: Mian M. Alauddin, Houston, TX (US); Uday Mukhopadhyay, Houston, TX (US); Juri Gelovani, Missouri City, TX (US); Ashutosh Pal, Houston, TX (US); William Bornmann, Missouri City, TX (US)

Correspondence Address: Nielsen IP Law LLC, 1177 West Loop South, Suite 1600, Houston, TX, 77027, US

(73) Assignee: Board of Regents, University of Texas System

(21) Appl. No.: 12/526,688

(22) PCT Filed: Feb. 11, 2008
(86) PCT No.: PCT/US08/53574

§ 371 Date: Apr. 21, 2010

Related U.S. Application Data
(60) Provisional application No. 60/889,477, filed on Feb. 12, 2007.

(51) Int. Cl. A61K 51/04; C07H 19/09; A61P 35/00

(52) U.S. Cl. 424/1.73; 536/28.53

(57) ABSTRACT

Compounds for in vivo diagnostic imaging of cellular proliferation are provided. The compounds include L-nucleosides such as a 2′-deoxy-2′-fluoro-L-ara-binofuranosyl pyrimidine nucleoside analogue. These nucleosides are labeled with a positron emitting radioisotope. The present invention also provides a method for in vivo diagnostic imaging of cellular proliferation.

### FIELD OF THE INVENTION

This present invention relates generally to PET-imaging agents. More specifically, the present invention relates to PET imaging agents that may be used to study cell proliferation.

### CROSS-REFERENCE TO RELATED APPLICATIONS

This application claims priority to U.S. Pat. App. Ser. No. 60/889,477 filed Feb. 12, 2007. The application is incorporated by reference herein in its entirety.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

Positron emission tomography, also called PET imaging or a PET scan, is the acquisition of physiologic images based on the detection of radioactive particles. PET provides for a diagnostic examination of biological processes, non-invasively, at the molecular level. Radioactive particles are typically emitted from radiolabeled PET imaging agents, nucleosides labeled with positron-emitting atoms such as positron-emitting halogens, which can incorporate into DNA and allow for the monitoring of cell proliferation. Images are then used to observe, for example, the synthesis of DNA in a tumor cell, and how rapidly the cell is dividing indicating the overall aggressiveness of the tumor.

In particular, PET imaging of cellular proliferation in vivo using radiolabeled nucleosides is helpful for understanding cancer. Unfortunately, commonly used nucleosides such as [¹³¹I]-UdR and [¹¹C]-thymidine suffer extensive catabolism following intravenous administration. Such chemical degradation events include dehalogenation, cleavage of the sugar from the base, and ring opening of the base. Thus, in vivo assessments require complex mathematical models to interpret kinetic data obtained in imaging studies.

Another common problem with radiolabeled nucleosides is the incorporation the catabolized byproducts into the DNA of the host after loss of the radiolabeled atom. For example, in the case of imaging studies with radiolabeled I-UdR using conventional techniques, it has been demonstrated that UdR formed after dehalogenation may be converted to TdR in mammalian systems and subsequently incorporated into DNA. Commerford, S. L. et al., Iododeoxyuridine Administered To Mice Is De-Iodinated And Incorporated Into DNA Primarily As Thymidylate, Biochem. Biophys. Res. Comm., 86: 112-118, 1979.

This metabolic pathway is likely related to the observed toxicity of the naturally occurring D-nucleosides. The toxicity of D-nucleosides is known in the art and strategies to alleviate the toxic side effects, for these otherwise valuable pharmaceuticals have been reported. See e.g., U.S. Pat. No. 5,756,478 at column 1, lines 11-14 and 28-33 and U.S. Published App. No. 2005/0250950 at page 1, paragraph 5, incorporated herein by reference in its entirety. Thus, a need exists for continued development of PET imaging agents that are minimally catabolized, and exhibit low toxicity to the host subject.

### SUMMARY OF THE INVENTION

The present invention provides radiolabeled, L-enantiomers, 2′-deoxy-2′-fluoro-L-arabinofuranosyl pyrimidine nucleoside analogues (referred to herein as “L nucleoside analogues radiolabeled”) and pharmaceutical compositions containing the same. The compounds of the subject invention, nucleosides labeled with a positron emitting radioisotope, are useful in connection with PET imaging and to determine the progress and rate of cancer proliferation. One preferred compound is 2′-deoxy-2′-fluoro-L-arabinofuranosyl pyrimidine nucleoside analogue radiolabeled (“[¹⁸F]-L-FMAU”).

The subject invention also provides a method for in vivo diagnostic imaging of cellular proliferation administering to a subject a 2′-deoxy-2′-fluoro-L-arabinofuranosyl pyrimidine nucleoside analogue to determine the rate and progress of a tumor. Detecting the positron emitting radioisotope in vivo localized in proliferating cells may be carried out under standard conditions.

The foregoing has outlined rather broadly the features of the present invention in order that the detailed description of the invention that follows may be better understood. Additional features and advantages of the invention will be described hereinafter, which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 shows the chemical structures of D-FMAU and L-FMAU.

FIG. 2 shows the synthesis of L-FMAU and [¹⁸F]L-FMAU.

FIG. 3 shows an HPLC purification of [¹⁸F]-L-FMAU.

FIG. 4 shows an HPLC chromatogram of [¹⁸F]-L-FMAU, co-injected with standard LFMAU

### DESCRIPTION OF THE INVENTION

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

2′-deoxy-L-fluoro-L-arabinofuranosyl pyrimidine nucleoside analogue radiolabeled (also referred to herein as “L nucleoside analogue”) includes compounds based on the common pyrimidine bases A (adenine), T (thymine), G (guanine), C (cytosine), and U (uracil). In accordance with the present invention, the L nucleoside analogue has shown a preferred uptake in proliferating cells compared to the enantiomeric D nuclesoside and therefore provides a superior PET imaging agent. Additionally, the L nucleoside is less toxic than the enantiomeric D nucleoside counterpart. A preferred example L nucleoside analogue disclosed herein is 2′-deoxy-2′-fluoro-L-arabinofuranosyluracil L-FMAU, in which, preferably fluorine carries the radiolabel, [¹⁸F]L-FMAU. One skilled in the art, however, will recognize the utility of radiolabeling other atoms, for example carbon.

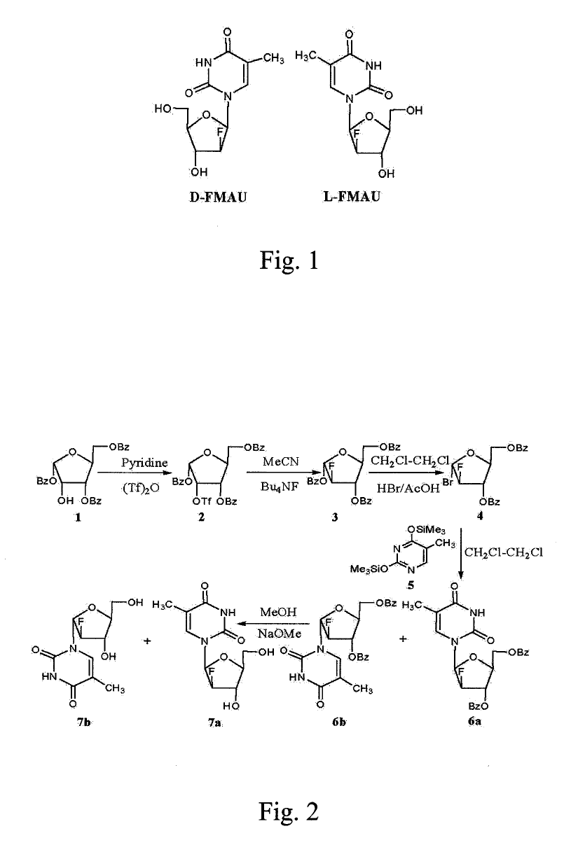

With reference to FIG. 1, L-nucleosides are the mirror image of naturally occurring D-nucleosides as exemplified by D-FMAU and L-FMAU. L-nucleosides are a novel class of compounds that exhibit antiviral and anticancer activities. Hu, R., et al., Behavior of Thymidylate Kinase Toward Monophosphate Metabolites and Its Role in the Metabolism of 1-(2′-Deoxy-2′-Fluoro-β-L-Arabinofuranosyl)-5-Methyluracil (Clevudine) and 2′,3′-Didehydro-2′,3′-Dideoxythymidine in Cells, Antimicrob Agents Chemother. 2005; 49: 2044-2049, hereafter ‘Hu’. Significantly, the unnatural L-nucleosides have lower toxicities compared to the natural D-nucleosides while maintaining high level of antiviral activity. Chu, C. K., et al., Use of 2′-Fluoro-5-Methyl-β-L-Arabinofuranosyluracil as a Novel Antiviral Agent for Hepatitis B Virus and Epstein-Barr Virus, Antimicrob Agents Chemother. 1995; 39: 979-981. Recognition of the reduced toxicity of L-nucleosides provides the impetus for the development of improved PET—imaging agents based on these enantiomeric nucleosides. Thus, the present invention provides compounds for in vivo diagnostic imaging of cellular proliferation based on a 2′-deoxy-2′-fluoro-L-arabinofuranosyl pyrimidine nucleoside analogue radiolabeled. The compounds are labeled with a positron emitting radioisotope, and the L-nucleoside analogue may be placed in a pharmaceutically acceptable carrier.

In particular the present invention provides an imaging agent which includes, but is not limited to 2′ deoxy-2′-[¹⁸F]-fluoro-5-methyl-1-β-L-arabinofuranosyluracil ([18F]-L-FMAU). 2′-Deoxy-2′-fluoro-5-methyl-1-β-L-arabinofuranosyl-uracil (L-FMAU) has been reported to have low cytotoxicity with potential antiviral activities against both hepatitis B virus (HBV) and Epstein-Barr virus but not human immunodeficiency virus Liu, S. H., et al., Unique Metabolism of a Novel Antiviral L-Nucleoside Analog, 2′-Fluoro-5-Methyl-β-L-Arabinofuranosyluracil: a Substrate for Both Thymidine Kinase and Deoxycytidine Kinase, Antimicrob. Agents Chemother, 1998, 42: 833-839.

L-FMAU (unlabeled) is reportedly an anti-HBV agent. See Hu and Lee, J., et al., Rapid Quantitative Determination of L-FMAU-TP from Human Peripheral-Blood Mononuclear Cells of Hepatitus B Virus-Infected Patients Treated With 1-FMAU by Ion-Pairing, Reverse-Phase, Liquid Chromatography/Electrospray Tandem Mass Spectrometry, The Drug Monit, 2006; 28: 131-137, hereafter ‘Lee’; Chong, Y., et al., Understanding the Unique Mechanism of L-FMAU (Clevudine) Against Hepatitis B Virus: Molecular Dynamics Study, Bioorg. Med. Chem. Lett. 2002; 12: 3459-3462, hereafter “Chong”. L-FMAU is mono-phosphorylated by cellular thymidine kinase (TK1), as well as deoxycytidine kinase (dCK) to its monophosphate, which then converted to the di- and tri-phosphate by the cellular di- and tri-phosphate nucleoside kinases (Pai, S. B., et al., Inhibition of Hepatitis B Virus by a Novel L-Nucleoside, 2′-Fluoro-5-Methyl-β-L-Arabinofuranosyl Uracil, Antimicrob. Agents Chemother. 1996; 40: 380-386, hereafter ‘Pai’; Chu, C. K., et al., Antivir. Therapy 1998; 3: 113-121, hereafter ‘Chu’. The triphosphate, L-FMAU-TP, is known to specifically inhibit viral DNA synthesis in a dose dependent manner without being incorporated into the infected cell DNA and does not cause DNA chain termination. See Lee, Pai, Chu, and Kocic, I., Current Opin. Investig. Drugs 2000; 1: 308-313. However, the precise mechanism of action of L-FMAU-TP at the polymerase level is not clearly understood (see Chong). L-FMAU is phosphorylated more efficiently than D-FMAU (see Pai and Chu). The phosphorylation of L-FMAU is mediated by both TK1 and dCK whereas the phosphorylation of D-FMAU is only regulated by one. Sherley, J. L., et al., Regulation of Human Thymidine Kinase During the Cell Cycle, J. Biol. Chem. 1988; 263: 8350-8358; Van der Wilt, C. L., et al., Expression of Deoxycytidine Kinase in Leukaemic Cells Compared With Solid Tumor Cell Lines, Liver Metastases and Normal Liver, Eur. J. Cancer 2003; 39: 691-697.

The imaging agent may be administered in a dosage unit form, such that a unit dose of the imaging agent is a non-toxic amount of the 2′-deoxy-2′-fluoro-L-arabinofuranosyl pyrimidine nucleoside analogue capable of localizing in proliferating cells and being detected in vivo. The specific activities of the radiolabeled 2′-deoxy-2′-fluoro-L-arabinofuranosyl pyrimidine nucleoside prepared as described herein below will generally range from about 1.5 to about 2.0 curies/micromole (Ci/μmol). Given that [¹⁸F] has a half life of about 110 minutes, a unit dose of may be in the range from about 100 to about 200 microcuries (μCi) [¹⁸]-L-FMAU, for example. (For comparison with new standards using Becquerel units (Bq), 37 Bq=1 nanocurie, thus there are 37 KBq to 1 microcurie.)

The present invention also provides a method for in vivo diagnostic imaging of cellular proliferation which includes administering to a subject in need thereof a 2′-deoxy-2′-fluoro-L-arabinofuranosyl pyrimidine nucleoside analogue radiolabeled. The imaging agent may be administered in accordance with procedures known in the art. For example, about 100 to about 200 μCi of radiolabeled material in physiological saline solution or equivalent vehicle along with any necessary adjuvant and other pharmaceutically acceptable carriers is administered intravenously to a subject prior to imaging or probe studies.

Next detecting the positron emitting radioisotope in vivo localized in proliferating cells is carried out by standard procedures. Data collection following administration may involve dynamic or static techniques with a variety of imaging devices, including PET cameras, gamma or SPECT (single photon emission computed tomography) cameras with either high energy collimators or coincidence detection capabilities, and probe devices designed to measure radioactive counts over specific regions of interest.

### Preparation of [¹⁸F]-L-FMAU

FIG. 2 represents the scheme for synthesis of the nucleoside, L-FMAU and [¹⁸F]-L-FMAU. Cold standard of L-FMAU was first synthesized following the synthetic scheme shown in FIG. 2. The radiosynthesis of [¹⁸F]-L-FMAU was performed according to the same scheme (FIG. 2) using [¹⁸F]-tetrabutylammonium fluoride, which was prepared in situ.

Compound 1 was synthesized in multiple steps following a previously reported method (Ma, T., et al., Structure-Activity Relationships of 1-(2-Deoxy-2-fluoro-β-L-arabino-furanosyl)pyrimidine Nucleosides as Anti-Hepatitis B Virus Agents, J. Med. Chem. 1996; 39: 2835-2843, hereafter ‘Ma’), and fully characterized by ¹H NMR and ¹³C NMR spectroscopy, and mass spectrometry. The ¹H NMR spectrum was consistent with the previous literature report (see Ma). Compound 1 was reacted with trifluoromethane sulfonic anhydride in pyridine to produce the triflate 2 in 94% yield. Compound 2 was characterized by ¹H NMR and ¹⁹F NMR spectroscopy. The ¹H NMR spectrum was identical with that reported in the literature for D-sugar (Tann, C. H., et al., Fluorocarbohydrates in Synthesis. An Efficient Synthesis of 1-(2-Deoxy-2-fluoro-β-D-arabino-furanosyl)-5-iodouracil (β-FIAU) and 1-(2-Deoxy-2-fluoro-μ-D-arabinofuranosyl)thymine (β-FMAU), J. Org. Chem. 1985; 50: 3644-3647, hereafter ‘Tann’), and the ¹⁹F NMR spectrum was also identical to that of the D-sugar as described in the experimental section. Fluorination of compound 2 was performed using tetrabutylammonium fluoride (Bu₄NF) in dry MeCN at 80° C. The fluorosugar 3 was characterized by ¹H NMR and ¹⁹F NMR spectroscopy, and the NMR spectra were identical with those of the D-sugar. See Tann and Van der Wilt, et al., Expression of Deoxycytidine Kinase in Leukaemic Cells Compared With Solid Tumour Cell Lines, Liver Metastases and Normal Liver, Eur. J. Cancer 2003; 39: 691-697.

Compound 4 was prepared by bromination of 3 with HBr/acetic acid following the literature method (see Tann) and as modified in our laboratory (see Alauddin). Compound 4 was used (without isolation) for the coupling reaction with thymine silyl ether 5. The unlabeled coupled products (6a and 6b) were isolated and characterized by NMR spectroscopy. Using the conventional analytical accessories, the ¹H and ¹⁹F NMR spectra were indistinguishable from those of the D-isomers (see Tann). Hydrolysis of the mixture (6a and 6b) produced 7a and 7b, and 7a was isolated by HPLC purification, and fully characterized by ¹H NMR and ¹⁹F NMR spectroscopy. ¹H NMR spectrum was consistent with that reported in the literature (see Ma), and ¹⁹F NMR spectrum (coupled) showed a multiplet centered at −200.87 ppm, which was identical to that of the D-isomer. Cold compound 7a (L-FMAU) was used as standard for HPLC analysis.

Radiofluorination of 2 was performed using [¹⁸]-n-Bu₄NF, which was prepared in situ from n-Bu₄HCO₃ and aqueous [¹⁸F]HF as reported earlier (see Alauddin). Radiochemical yields in the fluorination step were 75-87% (d.c.) with an average of 80% in 3 runs. This high yield fluorination was achieved by careful evaporation of the aqueous [¹⁸F]-n-Bu₄NF solution, which is extremely hygroscopic, however, is less stable under absolutely anhydrous condition. Unreacted fluoride was removed by passing the crude reaction mixture through a Sep-Pak cartridge (silica), and the crude product 3 was eluted with ethyl acetate.

[¹⁸F]-Labeled compound 4 was prepared following the literature method (see Tann) as modified in our laboratory (see Alauddin). The reaction was complete in 10 min at 80-82° C. Previously, we observed that compound 4 (D-sugar) is stable at higher temperature, however, a trace of acetic acid and water converts the compound 4 to 1-hydroxy and 1-acetoxy derivatives, respectively (see Alauddin). To avoid this decomposition, HBr and solvent were partially evaporated, then toluene (0.5 mL) was added to the reaction mixture, followed by evaporation to dryness. Acids are removed azeotropically at the initial stage of concentration, leaving the product unaffected. Crude 4 was found to be >90% radiochemically pure as previously reported HPLC method (see Alauddin) and used directly in the coupling reaction to reduce the synthesis time.

Coupling of the 1-bromo-2-fluorosugar 4 with thymine silyl ether 5 was performed as reported earlier (see Alauddin) with modification: the reaction temperature was raised from 100° C. to 120° C. and the reaction time was reduced from 60 min to 30 min. This reaction produced a mixture of protected nucleoside anomers 6a and 6b as observed previously during synthesis of D-FMAU (see Tann and Alauddin). Under these reaction conditions the overall yield in the synthesis remained quite similar. The thymine silyl ether 5 is susceptible to hydrolysis when exposed to air or moisture, and partial hydrolysis occurs as manifested by the appearance of solvent turbidity. Use of anhydrous solvent and inert atmosphere can avoid this decomposition and increase the yields. In general, an excess (4-5 equiv.) of thymine silyl ether is used in this coupling reaction, which compensates for the loss of thymine silyl ether due to decomposition, and the coupling reaction proceeds without significant reduction in yield, based on the amount of the ¹⁸F-fluorosugar produced.

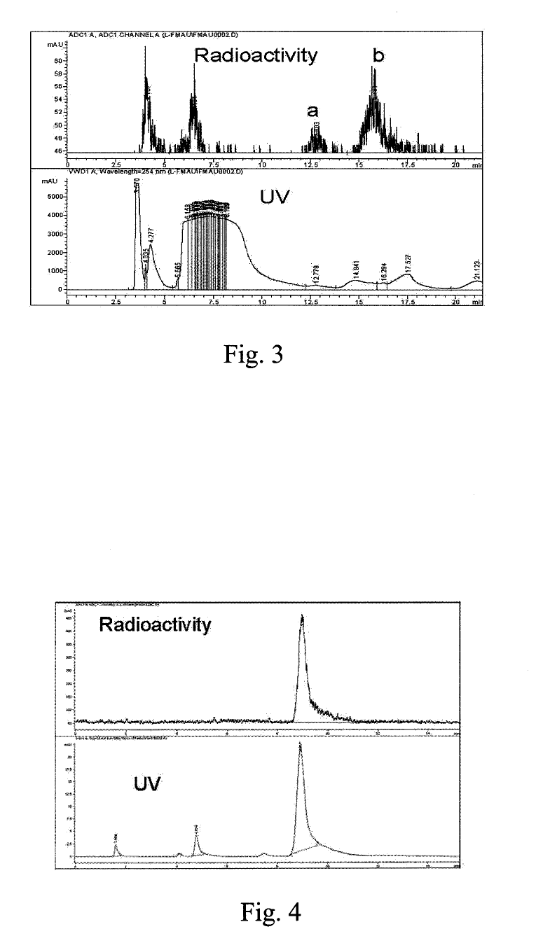

Compounds 7a and 7b were produced by basic hydrolysis of 6a and 6b, and 7a was isolated by HPLC purification as described in the experimental section. FIG. 3 shows a representative semi-preparative HPLC chromatogram of the crude reaction mixture. The radioactive peak at 15.7 min corresponds to the [¹⁸F]-L-FMAU, 7a, and the peak at 12.8 min is the α-anomer, 7b. The radiochemical purity of [¹⁸F]L-FMAU, which co-eluted on analytical HPLC with an authentic sample of unlabeled 7a, was >99% (FIG. 4).

The overall radiochemical yield of this synthesis was 24-28% (d.c.) from the end of bombardment (EOB) in four steps. The radiochemical purity of the final product, [¹⁸F]-L-FMAU, was >99% with an average calculated specific activity 2.2 Ci/μmole (81 GBq/μmole). The synthesis time was 3.3-3.5 hours from the EOB. In a typical synthesis, 7.8 mCi of labeled product was obtained starting from 100 mCi of [¹⁸F]-Fluoride.

### EXPERIMENTAL

All reagents and solvents were purchased from Aldrich Chemical Co. (Milwaukee, Wis.), and used without further purification. Solid phase extraction cartridge (Sep-Pak) was purchased from Waters Associates (Milford, Mass.). 2-Hydroxy-1,3,5-tri-O-benzoyl-α-L-ribofuranose 1, compound 2 and thymine-2,5-bis-trimethylsilyl ether 5 were prepared following literature methods. See Tann, Ma, and Alauddin, M. M., et al., Stereospecific Flourination of 1,3,5-tri-O-Benzoyl-α-D-Ribofuranose-2-Sulfonate Esters: Preparation of a Versatile Intermediate for Synthesis of 2′-[¹⁸F]-Fluoro-arabinonucleosides, J. Fluorine Chem. 2000; 106: 87-91, hereafter ‘Allaudin 2000’. Compounds 3 and 4 were not isolated, but used directly in the subsequent steps. The corresponding unlabeled compounds were characterized by ¹H and ¹⁹F NMR spectroscopy, and compared with those of the D-sugars.

Flash chromatography was performed using Merk silica gel 60 (mesh size 230-400 ASTM) or using an Isco (Lincon, Nebr.) combiFlash Companion or SQ16× flash chromatography system with RediSep columns (normal phase silica gel) and Fisher Optima TM grade solvents. Thin-layer chromatography (TLC) was performed on E.Merk (Darmstadt, Germany) silica gel F-254 aluminum-backed plates with visualization under UV (254 nm) and by staining with potassium permanganate or ceric ammonium molybdate.

Proton and ¹⁹F NMR spectra were recorded at the University of Texas MD Anderson Cancer Center on a Brucker 300 MHz or 600 MHz spectrometer using tetramethylsilane as an internal reference and hexafluorobenzene as an external reference, respectively. Low resolution mass spectral analysis was performed in house on HPLC-mass spectrometer (Applied Biosystem Q-Trap LC/MS/MS).

High performance liquid chromatography (HPLC) was performed on a 1100 series pump (Agilent, Germany), with built in UV detector operated at 254 nm, and a radioactivity detector with single-channel analyzer (Bioscan, Washington D.C.) using a semi-preparative C₁₈ reverse phase column (Alltech, Econosil, 10×250 mm, Deerfield, Ill.) and an analytical C₁₈ column (Rainin, Microsorb-MV, 4.6×250 mm, Emeryville, Calif.). An acetonitrile/water (MeCN/H₂O) solvent system (7.0% MeCN) was used for purification of the radiolabeled product and its quality control analysis.

### 2-Hydroxy-1,3,5-tri-O-benzoyl-α-L-ribofuranose 1

This compound was synthesized following a literature method reported by Ma et al. in multiple steps starting with the L-xylose (see Ma). The key intermediates and the desired compound 1 were characterized by spectroscopic methods and compared with those in the literature (see Ma). ¹H NMR spectrum of 1 was consistent with that previously reported in the literature (see Ma). ¹³C NMR (CDCl₃) δ: 166.1, 165.9, 165.4, 133.8, 133.7, 133.4, 129.9, 129.8, 129.7, 129.6, 129.5, 128.7, 128.6, 128.5, 95.9, 83.1, 72.2, 71.9, 64.1; MS: (C₂₆H₂₂0₈), calculated 462.1315, found 480.4 (M+NH₄).

### 2 Deoxy-2-trifluoromethanesulfonyl-1,3,5-tri-O-benzoyl-α-L-ribofuranose 2

This compound was prepared from compound 1 following a literature method reported earlier (see Tann and Alauddin 2000). Briefly, compound 1 (0.465 g, 1 mmol) was dissolved in anhydrous pyridine (7 mL) in a dry flask. The solution was cooled to 0° C. and trifluoromethane sulfonic anhydride (0.275 mL, 1.2 equiv.) was added slowly into the solution. The reaction mixture was stirred for 5 min at 0° C. then at room temperature for 1 hour. Pyridine was removed in vacuo and the crude product was purified by flash chromatography using a silica gel column and 20% acetone/hexane solvent system. The pure compound, 0.56 g was obtained in 94% yield. ¹H NMR (CDCl₃) δ: 8.03-8.16 (m, 6H, aromatic), 7.60-7.70 (m, 3H, aromatic), 7.40-7.53 (m, 6H, aromatic), 6.88 (d, 1H, C₁H, J=4.2 Hz), 5.79 (dd, 1H, J=9.6 Hz, J=3.3 Hz), 5.56 (dd, 1H, J=10.8 Hz, J=4.5 Hz), 4.88 (q, 1H, C₄H, J=3.3 Hz), 4.73 (dd, 2H, C₅H, J=51.9 Hz, J=2.7 Hz), ¹⁹F NMR (CDCl₃) δ: −74.45.

### 2 Deoxy-2 fluoro-1,3,5-tri-O-benzoyl-α-L-arabinofuranose 3

Both cold and radiosynthesis were performed according to the synthetic scheme. Only radiosynthesis is described here. Compound 3 was prepared following the method developed in our laboratory with modification (see Alauddin and Alauddin 2000). Briefly, the aqueous [¹⁸F]fluoride was trapped in anion exchange cartridge (ABX, Germany) and eluted with a solution of n-Bu₄NHCO₃ (400 μL, 1% by wt.) into a v-vial, and the solution evaporated azeotropically with acetonitrile (1.0 mL) to dryness at 79-80° C. under a stream of argon. To the dried [¹⁸F]-n-Bu₄NF, a solution of 2 (5.0 mg) in anhydrous acetonitrile (0.5 mL) was added, and the mixture was heated at 80° C. for 20 min. The reaction mixture was cooled to room temperature, passed through a Sep-Pak cartridge (silica gel), and eluted with ethyl acetate (2.0 mL). After evaporation of solvent with a stream of argon at 80° C., the residue was used for the next step without further purification.

### 1-Bromo-2-deoxy-2-[¹⁸F]fluoro-3,5-di-O-benzoyl-α-L-arabinofuranose 4

The radiolabeled fluorosugar 3 was dissolved in 1,2-dichloroethane (0.4 mL) under argon. Hydrogen bromide (HBr) in acetic acid (30%, 0.1 mL) was added, and the reaction mixture was heated for 10 minutes at 80-82° C. The reaction mixture was partially evaporated then diluted with toluene (0.5 mL) and continued evaporation to dryness under a stream of argon. The dry crude product was used for the coupling experiment without purification.

### 2′ Deoxy-2′-[¹⁸F]fluoro-3′,5′-di-O-benzoyl-5-methyl-1β/α-L-arabinofuranosyluracil 6a/6b

To 1-bromo-2-deoxy-2-[¹⁸F]fluoro-3,5-di-O-benzoylarabinofuranose 4 as obtained in the previous step was added a solution of freshly prepared 2,4-bis-β-(trimethylsilyl)thymine 5 (35 mole, 4 equiv.) in 1,2-dichloroethane (0.5 mL). The vial was heated in a heating block at 120° C. for 30 minutes. The reaction mixture was cooled to room temperature, the solvent was evaporated at 80° C. under a stream of argon and the crude product was hydrolyzed as described in the next step.

### 2′ Deoxy-2′ [¹⁸F]fluoro-5-methyl-1β-L-arabinofuranosyluracil 7a

The crude mixture of 2′-deoxy-2′-[¹⁸F]fluoro-3,5-di-O-benzoyl-5-methyl-1-β-L-arabinofuranosyluracil (6a and 6b) was dissolved in methanol (0.3 mL). Sodium methoxide (0.5M solution in methanol, 0.1 mL) was added, and the mixture was heated for 5 minutes at reflux. The reaction mixture was cooled and neutralized with HCl (1N in methanol, 0.1 mL). After evaporation of methanol, the crude material was diluted with HPLC solvent and purified by HPLC using 7.0% MeCN/H₂O at a flow of 4.0 mL/minute. The appropriate fraction specified by the prior determined retention time of an authentic sample eluted at around 15.5 min (FIG. 3) was collected and evaporated to dryness. The pure product was re-dissolved in saline (Abbott Lab., Chicago, Ill.) and filtered through a 0.22 micron filter (Millipore, Bedford, Mass.). An aliquot of the final product was analyzed by analytical HPLC, and found to be co-eluting with the authentic compound (FIG. 4).

### Example PET Imaging

### Example 1

### Comparative microPET Imaging with [¹⁸F]-FLT, [¹⁸F]-D-FMAU and [¹⁸F]L-FMAU for Detection of Proliferative Activity of Tumors in Mice Bearing Human Nsclc Xenografts

This study aimed to assess the efficacy of PET imaging of tumor proliferative activity with [¹⁸F]-3′-deoxy-3′-fluorothymidine ([¹⁸F]-FLT), [¹⁸F]-D-FMAU and [¹⁸F]-L-FMAU in human NSCLC xenografts.

All radiotracers were synthesized following literature methods. Two human NSCLC cell lines, H441 (rapidly growing) and H3255 (slow growing) were used to grow (s.c.) tumor xenografts in nude mice. Eight nu/nu mice were injected with 3×10⁶ cells (H3255) onto right shoulder, and 3 weeks later, 3×10⁶ cells (H441) were injected onto opposite shoulder. When tumors grew approximately 1 cm in diameter, PET imaging with [¹⁸F]-FLT, [¹⁸F]-D-FMAU [¹⁸F]-L-FMAU were performed on three consecutive days. Each animal received 3.7 MBq of radiotracer via the tail vein, then dynamic scan was performed using micro-PET up to 2 hours of post-injection.

Tumor uptake and tumor-to-muscle (T/M) ratios of [¹⁸F]-FLT, [¹⁸F]-D-FMAU and [¹⁸F]L-FMAU for different tumors are summarized in Table 1. At 2 h post-injection, the highest tumor uptake was observed with [¹⁸F]-D-FMAU both in H441 and H3255, which indicates higher sensitivity for detection of thymidine kinase activity inside these tumors. On the other hand, tumor-to muscle ratio of [¹⁸F]-FLT accumulation was higher than that for [¹⁸F]-D-FMAU and [¹⁸F]-L-FMAU. Although the tumor uptake of [¹⁸F]-L-FMAU was lower than that of [¹⁸F]-D-FMAU, it showed higher T/M ratio in H441 tumors.

TABLE 1

| % ID/g | [¹⁸F]-FLT | [¹⁸F]-D-FMAU | [¹⁸F]-L-FMAU |
|---|---|---|---|
| H441 | 5.10 ± 1.45 | 7.74 ± 1.39 | 3.13 ± 1.11 |
| H3255 | 0.57 ± 0.33 | 4.49 ± 1.08 | 1.38 ± 0.81 |
| T/M ratio | [¹⁸F]-FLT | [¹⁸F]-D-FMAU | [¹⁸F]-L-FMAU |
| H441 | 12.94 ± 4.38 | 3.37 ± 1.19 | 4.15 ± 1.82 |
| H3255 | 1.50 ± 0.90 | 1.96 ± 0.74 | 1.62 ± 0.50 |

### Example 2

### Micro-PET Imaging of Tumor Proliferative Activity with [¹⁸F]-FLT, [¹⁸F]-D-FMAU and [¹⁸F]-L-FMAU in Nude Mice Bearing Human Colon Cancer Xenografts

This study aimed to assess the efficacy of PET imaging of tumor proliferative activity with [18F]-FLT, [¹⁸F]-D-FMAU and [¹⁸F]-L-FMAU in human colon cancer xenografts.

The radiotracers, [¹⁸F]-FLT, [¹⁸F]-D-FMAU and [¹⁸F]-L-FMAU, were synthesized according literature methods in high specific activity. Human colon cancer cells, Colo205 were used to grow tumor xenografts in nude mice. Four nu/nu mice were injected (s.c.) with 3×10⁶ cells onto the shoulder. When tumors were approximately 1 cm in diameter, PET imaging with [¹⁸F]-FLT, [¹⁸F]-D-FMAU [¹⁸F]-L-FMAU were performed on three consecutive days using one tracer at a time. Each animal received 3.7 MBq of radiotracer via the tail vein, then dynamic imaging was performed using micro-PET up to 2 hours of post-injection.

Tumor uptake and tumor-to-muscle (T/M) ratios of [¹⁸F]-FLT, [¹⁸F]-D-FMAU and [¹⁸F]-L-FMAU are summarized in Table 2. At 2 h post-injection, the highest tumor uptake was observed with [¹⁸F]-FLT, and less with [¹⁸F]-D-FMAU and [¹⁸F]-FMAU, which were comparable. However, tumor-to muscle ratio of [¹⁸F]-FLT accumulation was lower than that of [¹⁸F]-D-FMAU and [¹⁸F]-L-FMAU. [¹⁸F]-L-FMAU had higher uptake and tumor/muscle ratio compared to the D-[¹⁸F]-FMAU, which is probably due to higher resistance to metabolic degradation.

TABLE 2

| | [¹⁸F]-FLT | [¹⁸F]-D-FMAU | [¹⁸F]-L-FMAU |
|---|---|---|---|
| % ID/g | 5.20 ± 1.32 | 2.37 ± 0.58 | 2.93 ± 0.75 |
| | [¹⁸F]-FLT | [¹⁸F]-D-FMAU | [¹⁸F]-L-FMAU |
| T/M ratio | 1.51 ± 0.61 | 1.95 ± 0.37 | 2.85 ± 0.89 |

PET with [¹⁸F]-L-FMAU provides more specific images of proliferative activity in colon carcinomas, as compared to [¹⁸F]-FLT and D-[¹⁸F]-FMAU. Higher tumor/muscle ratio of [¹⁸F]-L-FMAU compared to D-[¹⁸F]-FMAU, and [¹⁸F]-L-FMAU demonstrates certain advantages over [¹⁸F]-D-FMAU for PET imaging of tumor proliferative activity.

Advantageously, the present invention provides radiolabeled L-nucleoside analogues which may exhibit lower toxicity than their D-nucleoside counterpart. Additionally, such compounds may be more robust against catabolic processes that compromise the structural integrity and effectiveness of the imaging agent. Additionally, such imparted stability may obviate the need for complex computational modeling currently in use in typical PET imaging procedures. [¹⁸F]-FMAU as disclosed herein is merely representative of L-nucleosides analogues that may be used as PET imaging agents. One skilled in the art will recognize that other L-nucleosides may be useful and fall within the scope of the present invention, including for example, radiolabeled 2′-Deoxy-2′-fluoro-5-fluoro-1-β-D-arabinofuranosyluracil (L-FFAU), 2′-fluoro-5-iodo-1-O-L-arabinofuranosyluracil (L-FIAU), and 5-iodo-1-(2-deoxy-2-fluoro-L-arabinofuranosyl)cystosine (L-FIAC).

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed:

1. A compound comprising a 2′-deoxy-L-fluoro-L-arabinofuranosyl pyrimidine analogue radiolabeled.

2. The compound of claim 1 wherein, said 2′-deoxy-2′-fluoro-L-arabinofuranosyl pyrimidine is [¹⁸F]-L-FMAU.

3. A pharmaceutical composition for in vivo diagnostic imaging of cellular proliferation comprising:
2′-deoxy-2′-fluoro-L-arabinofuranosyl pyrimidine nucleoside analogue labeled with a positron emitting radioisotope; and
a pharmaceutically acceptable carrier

4. The pharmaceutical composition of claim 3, wherein the imaging agent is 2′ deoxy-2′-[¹⁸F]-fluoro-5-methyl-1-O-L-arabinofuranosyluracil ([¹⁸F]-FMAU).

5. A method for in vivo diagnostic imaging of cellular proliferation comprising:
administering to a subject in need thereof a compound comprising 2′-deoxy-2′-fluoro-L-arabinofuranosyl pyrimidine nucleoside analogue radiolabeled; and
detecting the positron emitting radioisotope in vivo localized in proliferating cells.

6. The method of claim 5, wherein the positron emitting radioisotope is [¹⁸F].

7. The method of claim 5, wherein [¹⁸F] is administered in a unit dose from about 100 microcuries to about 200 microcuries.

8. The method of claim 5, wherein the compound is 2′-deoxy-2′-[¹⁸F]-fluoro-5-methyl-1-β-L-arabinofuranosyluracil ([18F]-FMAU).

9. A compound with IUPAC designation 2′-deoxy-2′-[¹⁸F]-fluoro-5-methyl-1-β-L-arabinofuranosyluracil ([18F]-FMAU).
